(19) United States
(12) Patent Application Publication
(10) Pub. No.: US 2015/0126831 A1
(43) Pub. Date: May 7, 2015

(54) MEDICAL SENSOR WITH AMBIENT LIGHT SHIELDING

(71) Applicant: COVIDIEN LP, Mansfield, MA (US)

(72) Inventors: Donald R. Sandmore, Lyons, CO (US); Alissa K. Wong, Superior, CO (US)

(21) Appl. No.: 14/531,627

(22) Filed: Nov. 3, 2014

Related U.S. Application Data
(60) Provisional application No. 61/899,510, filed on Nov. 4, 2013.

(51) Int. Cl. A61B 5/1455; A61B 5/08; A61B 5/024

(52) U.S. Cl. CPC A61B 5/14552; A61B 5/02427; A61B 5/0816
USPC 600/323; 600/476

(57) ABSTRACT

Medical sensors assemblies with shielding elements are provided that may be applied to a sensor body to reduce ambient light infiltration. In one embodiment, the shielding elements may be applied only about a portion of the sensor body such that an additive pressure of the shielding element and the sensor body on the patient's tissue is reduced. For example, the shielding element may include openings that reduce the total surface area of the sensor body that is covered by the shielding element. In addition, such shielding elements may be provided as separate accessories for a sensor that may be applied only when ambient light infiltration is detected.

### CROSS-REFERENCE TO RELATED APPLICATION

This application claims the benefit of Provisional Application Ser. No. 61/899,510, filed Nov. 4, 2013, entitled “MEDICAL SENSOR WITH AMBIENT LIGHT SHIELDING”, which is incorporated by reference herein in its entirety.

### BACKGROUND

The present disclosure relates generally to medical sensors and, more particularly, to medical sensors with ambient light shielding.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of devices have been developed for non-invasively monitoring physiological characteristics of patients. For example, a pulse oximetry sensor system may non-invasively detect various patient blood flood characteristics, such as the blood-oxygen saturation of hemoglobin in arterial blood, the volume of individual blood pulsations supply the tissue, and/or the rate of blood pulsations corresponding to each heart beat of a patient. During operation, the pulse oximeter sensor emits light and photoelectrically senses the absorption and/or scattering of the light after passage through the perfused tissue. A photo-plethysmographic waveform, which corresponds to the cyclic attenuation of optical energy through the patient's tissue, may be generated from the detected light. Additionally, one or more physiological characteristics may be calculated based upon the amount of light absorbed or scattered. More specifically, the light passed through the tissue may be selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

For example, a reflectance-type sensor placed on a patient's forehead may emit light into the site and detect the light that is “reflected” back after being transmitted through the forehead region. A transmission-type sensor having a bandage configuration may be placed on a finger, wherein the light waves are emitted through and detected on the opposite side of the finger. In either case, the amount of light detected may provide information that corresponds to valuable physiological patient data. The data collected by the sensor may be used to calculate one or more of the above physiological characteristics based upon the absorption or scattering of the light. For instance, the emitted light is typically selected to be of one or more wavelengths that are absorbed or scattered in an amount related to the presence of oxygenated versus de-oxygenated hemoglobin in the blood. The amount of light absorbed and/or scattered may be used to estimate the amount of the oxygen in the tissue using various algorithms.

When the sensor is applied to the patient, it is generally desirable that the sensor conform to the underlying tissue, fitting snugly. Such a snug fit helps exclude environmental or ambient light, which might otherwise produce incorrect or erroneous physiological data. However, a too-snug sensor may be uncomfortable in some circumstances and/or may potentially compromise the accuracy of physiological measurements. Further, a sensor applied with adhesive that is frequently repositioned may not adhere as well after being moved several times.

### SUMMARY

In one embodiment, a sensor assembly is provided. The sensor assembly includes a sensor body comprising a first lobe and a second lobe coupled by a neck; an emitter disposed on a patient-contacting side of the sensor body on the first lobe and configured to transmit light into a patient tissue; a first detector disposed on the patient-contacting side of the sensor body on the second lobe and configured to detect a first portion of light passing through the patient tissue; a second detector disposed on the patient-contacting side of the sensor body on the second lobe and configured to detect a second portion of light passing through the patient tissue, wherein the first detector is closer to the emitter than the second detector; and an adhesive layer disposed about at least a portion of a perimeter of the sensor body. The sensor assembly also includes a shielding element configured to be adhered to the sensor body on a surface opposing the patient-contacting surface and to the adhesive layer, wherein the shielding element, when adhered to the sensor body, extends beyond the perimeter of the sensor body and does not cover the sensor body in an area corresponding to the emitter, the first detector, and the second detector.

In another embodiment, a sensor assembly is provided that includes a sensor body; an emitter disposed on a patient-contacting side of the sensor body; at least one detector disposed on the patient-contacting side of the sensor body and configured to detect a first portion of light passing through the patient tissue; and a shielding element comprising an adhesive surface configured to be adhered in part to a patient's tissue and in part to the sensor body on a surface opposing the patient-contacting surface layer, wherein the shielding element, when adhered to the sensor body, applies pressure unevenly across the sensor body such that a pressure applied to the patient's tissue by the emitter and the at least one detector is less than a pressure applied by a perimeter of the sensor body.

In another embodiment, a method is provided that includes the steps of applying a sensor to a patient's tissue wherein the sensor comprises a sensor body having a first lobe and a second lobe coupled by a neck such that a first optical element disposed on the first lobe and a second optical element disposed on the second lobe are in direct contact with the patient's tissue; receiving an indication that measurements acquired by the sensor are associated with ambient light infiltration; and applying a shielding element in part to the sensor body on a surface opposing a patient-contacting surface and in part to the patient's tissue in an area about a perimeter of the sensor body such that the shielding element, when adhered to the sensor body, extends beyond the perimeter of the sensor body and does not cover the sensor body in an area corresponding to the first optical element and the second optical element, wherein the applying of the shielding element is only when the measurements acquired by the sensor are associated with ambient light infiltration.

### BRIEF DESCRIPTION OF THE DRAWINGS

Advantages of the disclosed techniques may become apparent upon reading the following detailed description and upon reference to the drawings in which:

FIG. 1 is a front view of an embodiment of a monitoring system configured to be used with a sensor for regional saturation, in accordance with an aspect of the present disclosure;

FIG. 2 is a block diagram of the monitoring system of FIG. 1, in accordance with an aspect of the present disclosure;

FIG. 3 is a top view of an embodiment of a sensor kit including a sensor and one or more shielding elements, in accordance with an aspect of the present disclosure;

FIG. 4 is a side view of a shielding element being applied to a sensor on a non-tissue contacting surface, in accordance with an aspect of the present disclosure;

FIG. 5 is a top view of a shielding element applied to a sensor on a non-tissue contacting surface, in accordance with an aspect of the present disclosure;

FIG. 6 is a top view of a shielding element, in accordance with an aspect of the present disclosure;

FIG. 7 is a top view of a tissue contacting surface of the sensor of FIG. 6;

FIG. 8 is a side view of a shielding element retained in association with a sensor cable, in accordance with an aspect of the present disclosure; and

FIG. 9 is a flow diagram of a method of determining if a shielding element should be applied to a sensor, in accordance with an aspect of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. It should also be understood that elements of the disclosed embodiments may be combined together or exchanged with one another.

Plethysmography sensors are typically placed on a patient in a location that is normally perfused with blood to facilitate measurement of the desired blood characteristics, such as arterial oxygen saturation measurement, regional saturation, respiration rate, etc. The most common sensor sites include a patient's fingertips, toes, earlobes, or forehead. Regardless of the placement of a sensor used, the reliability of the plethysmography measurement is related to the accurate detection of transmitted light that has passed through the perfused tissue and that has not been supplemented by undesired light sources. Such supplementation and/or modulation of the signals transmitted to a monitor by the sensor can cause variability in the resulting measurements. The contribution of ambient and/or shunted light may affect the measurement of the particular blood constituent.

Provided herein are sensor assemblies that include light shielding elements that are separate from the sensor body such that a caregiver may choose to apply the shielding elements when and if ambient light infiltration is an issue for a particular sensor. In certain embodiments, the shielding elements are configured such that the supplemental shielding is achieved without having to remove or adjust the placement of an already-applied sensor. In addition, the shielding elements components are configured to shield a perimeter of the sensor body from ambient light infiltration without adding additional pressure to the optical components that may interfere with measurement accuracy.

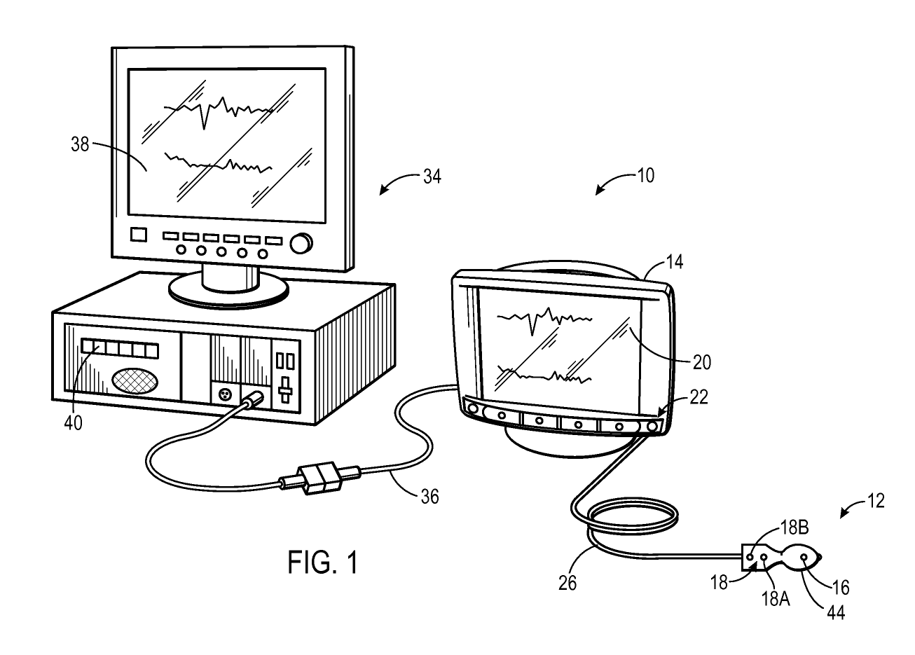

The shielding elements as disclosed may be used in conjunction with sensor and sensors assemblies and a patent monitoring system. With this in mind, FIG. 1 depicts an embodiment of a patient monitoring system 10 that may be used in conjunction with a medical sensor 12 including one or more light shielding components as provided herein. Although the depicted embodiments relate to sensors for use on a patient's head, it should be understood that, in certain embodiments, the features of the sensor 12 as provided herein may be incorporated into sensors for use on other tissue locations, such as the back, the stomach, the heel, the ear, a digit, an arm, a leg, or any other appropriate measurement site. In addition, although the embodiment of the patient monitoring system 10 illustrated in FIG. 1 relates to photoplethysmography, the system 10 may be configured to obtain a variety of medical measurements with a suitable medical sensor. In one embodiment, the sensor 10 may be a pulse oximetry sensor or regional oxygen saturation sensor.

For example, the sensors described herein may incorporate one or more emitters and one or more detectors for determining the level of blood oxygen saturation in a particular region, such as a cerebral or somatic region. By way of example, an INVOS® cerebral/somatic sensor, such as an OxyAlert™ NIR sensor or a SomaSensor® by Covidien which may include one or more emitters and a pair of detectors for determining site-specific oxygen levels, represents such sensors. Moreover, other types of sensors, such as those used for measuring water fraction, hematocrit, etc., may benefit from the techniques disclosed herein as well. For example, the system 10 may additionally be configured to determine patient electroencephalography (e.g., a bispectral index), or any other desired physiological parameter such as water fraction or hematocrit.

As noted, the system 10 includes the sensor 12 that is communicatively coupled to a patient monitor 14. The illustrated sensor 12 includes an emitter 16 and a pair of detectors 18. The emitter 16 and detectors 18 of the sensor 12 are coupled to the monitor 14 via one or more cables 26 coupled to a sensor port of the monitor 14. The monitor 14 includes a monitor display 20 configured to display information regarding the physiological parameters monitored by the sensor 12, information about the system, and/or alarm indications. The monitor 14 may include various input components 22, such as knobs, switches, keys and keypads, buttons, etc., to provide for operation and configuration of the monitor. The monitor 14 also includes a processor that may be used to execute code such as code for implementing various monitoring functionalities enabled by the sensor 12. As discussed below, for example, the monitor 14 may be configured to process signals generated by the detectors 18 to estimate the amount of oxygenated vs. de-oxygenated hemoglobin in a monitored region of the patient.

The monitor 14 may be any suitable monitor, such as a pulse oximetry monitor available from Covidien or an INVOS® System monitor also available from Covidien. Furthermore, to upgrade conventional operation provided by the monitor 14 to provide additional functions, the monitor 14 may be coupled to a multi-parameter patient monitor 34 via a cable 36 connected to a sensor input port or via a cable 36 connected to a digital communication port. In addition to the monitor 14, or alternatively, the multi-parameter patient monitor 34 may be configured to calculate physiological parameters and to provide a central display 38 for the visualization of information from the monitor 14 and from other medical monitoring devices or systems. The multi-parameter monitor 34 includes a processor that may be configured to execute code. The multi-parameter monitor 34 may also include various input components 40 such as knobs, switches, keys and keypads, buttons, etc., to provide for operation and configuration of the a multi-parameter monitor 34. In addition, the monitor 14 and/or the multi-parameter monitor 34 may be connected to a network to enable the sharing of information with servers or other workstations.

The sensor 12, illustrated as operatively connected to the monitor 14, may include a sensor body 44 that houses the emitter 16 for emitting light at certain wavelengths into a tissue of a patient and the detectors 18 for detecting the light after it is reflected and/or absorbed by the blood and/or tissue of the patient. The sensor body 44 may be formed from any suitable material, including rigid or conformable materials, such as fabric, paper, rubber or elastomeric compositions (including acrylic elastomers, polyimide, silicones, silicone rubber, celluloid, PMDS elastomer, polyurethane, polypropylene, acrylics, nitrile, PVC films, acetates, and latex). In certain embodiments, the sensor 12 may be a wireless sensor 12. Accordingly, the wireless sensor 12 may establish a wireless communication with the patient monitor 14 and/or the multi-parameter patient monitor 34 using any suitable wireless standard. By way of example, the wireless module may be capable of communicating using one or more of the ZigBee standard, WirelessHART standard, Bluetooth standard, IEEE 802.11x standards, or MiWi standard.

As illustrated in FIG. 1 the sensor 12 may include the emitter 16 and the two detectors 18: one detector 18A that is relatively “close” to the emitter 16 and another detector 18B that is relatively “far” from the emitter 16. Light intensity of one or more wavelengths may be received at both the “close” and the “far” detectors. Thus, the detector 18A may receive a first portion of light and the detector 18B may receive a second portion of light. Each of the detectors 18 may generate signals indicative of their respective portions of light. For example, the resulting signals may be contrasted to arrive at a regional saturation value that pertains to additional tissue through which the light received at the “far” detector passed (tissue in addition to the tissue through which the light received by the “close” detector passed, e.g., the brain tissue) when it was transmitted through a region of a patient (e.g., a patient's cranium). Surface data from the skin and skull is subtracted out to produce an rSO₂ value for deeper tissues.

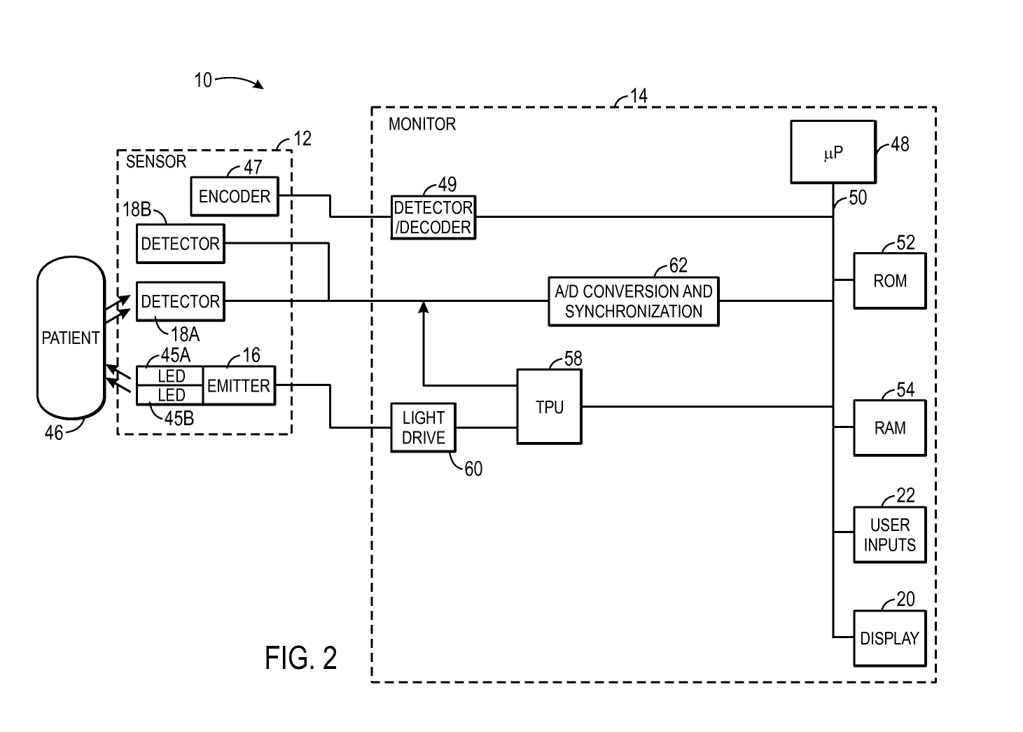

Turning to FIG. 2, a simplified block diagram of the medical system 10 is illustrated in accordance with an embodiment. The sensor 12 may include optical components in the forms of the emitter 16 and detectors 18. The emitter 16 and the detectors 18 may be arranged in a reflectance or transmission-type configuration with respect to one another. However, in embodiments in which the sensor 12 is configured for use on a patient's forehead, the emitter 16 and detectors 18 may be in a reflectance configuration. An emitter 16 may also be a light emitting diode, superluminescent light emitting diode, a laser diode, or a vertical cavity surface emitting laser (VCSEL). An emitter 16 and the detectors 18 may also include optical fiber sensing elements. An emitter 16 may include a broadband or “white light” source, in which case the detectors 18 could include any of a variety of elements for selecting specific wavelengths, such as reflective or refractive elements or interferometers. These kinds of emitters and/or detectors would typically be coupled to the sensor 12 via fiber optics. Alternatively, the sensor 12 may sense light detected from the tissue is at a different wavelength from the light emitted into the tissue. Such sensors may be adapted to sense fluorescence, phosphorescence, Raman scattering, Rayleigh scattering and multi-photon events, or photoacoustic effects. In one embodiment, the emitter 16 may be configured for use in a regional saturation technique. To that end, the emitter 16 may include two light emitting diodes (LEDs) 45A and 45B that are capable of emitting at least two wavelengths of light, e.g., red or near infrared light. In one embodiment, the LEDs 45A and 45B emit light in the range of 600 nm to about 1000 nm. In a particular embodiment, the one LED 45A is capable of emitting light at 730 nm and the other LED 45B is capable of emitting light at 810 nm. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the sensor 12, the detectors 18A and 18B may be an array of detector elements that may be capable of detecting light at various intensities and wavelengths. In one embodiment, light enters the detector 18 (e.g., detector 18A or 18B) after passing through the tissue of the patient 46. In another embodiment, light emitted from the emitter 16 may be reflected by elements in the patient's tissue to enter the detector 18. The detector 18 may convert the received light at a given intensity, which may be directly related to the absorbance and/or reflectance of light in the tissue of the patient 46, into an electrical signal. That is, when more light at a certain wavelength is absorbed, less light of that wavelength is typically received from the tissue by the detector 18, and when more light at a certain wavelength is reflected, more light of that wavelength is typically received from the tissue by the detector 18. After converting the received light to an electrical signal, the detector 18 may send the signal to the monitor 14, where physiological characteristics may be calculated based at least in part on the absorption and/or reflection of light by the tissue of the patient 46.

The medical sensor 12 may also include an encoder 47 configured to provide signals indicative of the wavelength of one or more light sources of the emitter 16, which may allow for selection of appropriate calibration coefficients for calculating a physical parameter such as blood oxygen saturation. The encoder 47 may, for instance, include a coded resistor, an electrically erasable programmable read only memory (EEPROM), or other coding device (such as a capacitor, inductor, programmable read only memory (PROM), RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. Further, the encoder 47 may include encryption coding that prevents a disposable part of the medical sensor 12 from being recognized by a microprocessor 48 unable to decode the encryption. For example, a detector/decoder 49 may translate information from the encoder 47 before the processor 48 can properly handle it. In some embodiments, the encoder 47 and/or the detector/decoder 49 may not be present.

Signals from the detector 18 and/or the encoder 47 may be transmitted to the monitor 14. By way of example, the monitor 14 shown in FIG. 2 may be an INVOS® System monitor 14 available from Covidien. The monitor 14 may include one or more processors 48 coupled to an internal bus 50. Also connected to the bus 50 may be a ROM memory 52, a RAM memory 54, and the display 20. A time processing unit (TPU) 58 may provide timing control signals to light drive circuitry 60, which controls when the emitter 16 is activated, and if multiple light sources are used, the multiplexed timing for the different light sources. The received signal from the detector 18 may be passed through analog-to-digital conversion and synchronization 62 under the control of timing control signals from the TPU 58. Specifically, the signal may undergo synchronized demodulation and optionally amplification and/or filtering. For example, the LEDs 45A and 45B may be driven out-of-phase, sequentially and alternatingly with one another (i.e., only one of the LEDs 45A and 45B being driven during the same time interval) such that the detector 18 receives only resultant light spectra emanating from one LED at a time. Demodulation of the signal enables the data associated with the LEDs 45A and 45B to be distinguished from one another. After demodulation, the digital data may be downloaded to the RAM memory 54.

In an embodiment, based at least in part upon the received signals corresponding to the light received by detector 18, the processor 48 may calculate the physiological parameter, in this case regional saturation, using various algorithms. These algorithms may use coefficients, which may be empirically determined. For example, algorithms relating to the distance between an emitter 16 and various detector elements in a detector 18 may be stored in the ROM memory 52 and accessed and operated according to processor 48 instructions.

Furthermore, one or more functions of the monitor 14 may also be implemented directly in the sensor 12. For example, in some embodiments, the sensor 12 may include one or more processing components capable of calculating the physiological characteristics from the signals obtained from the patient 46. In accordance with the present techniques, the sensor 12 may be configured to provide desired contact between the patient 46 and the detector 18, and/or the emitter 16. The sensor 12 may have varying levels of processing power, and may output data in various stages to the monitor 14, either wirelessly or via the cable 26 (see FIG. 1). For example, in some embodiments, the data output to the monitor 14 may be analog signals, such as detected light signals (e.g., oximetry signals or regional saturation signals), or processed data.

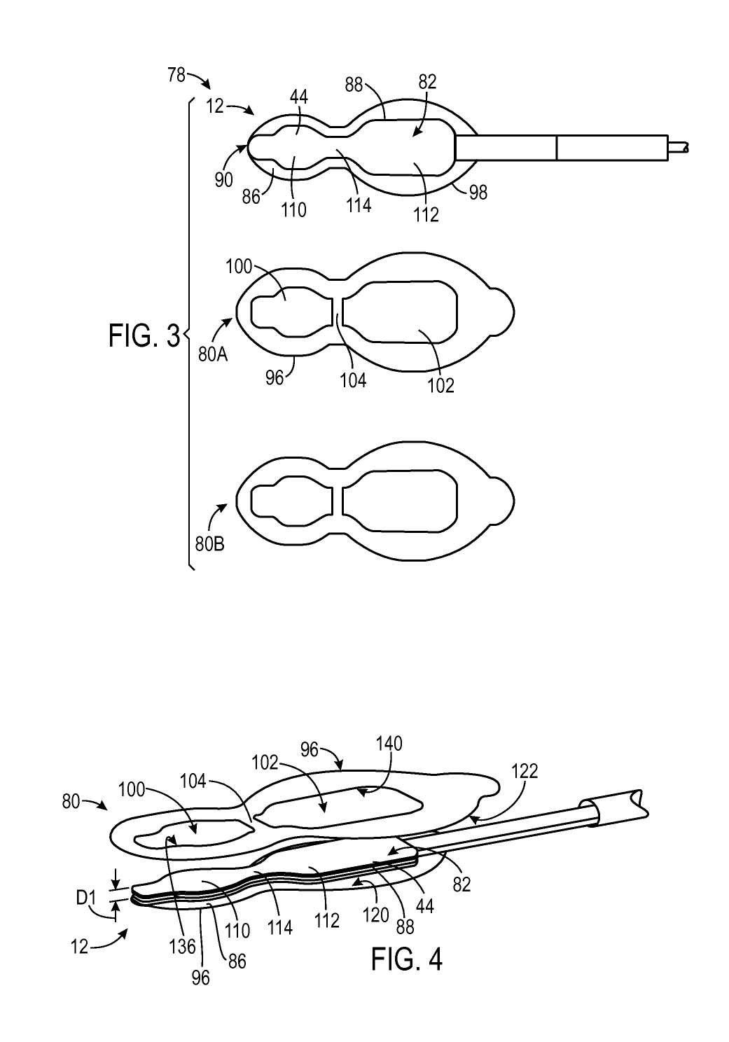

The shielding elements provided herein may be part of a sensor kit or assembly 78 that includes one or more shielding elements 80, illustrated as shielding elements 80a and 80b in FIG. 3. For example, an individual sensor 12 may be provided or packaged with one or more shielding elements 80 that are sized and shaped to be used in conjunction with the sensor 12. In one embodiment, a pediatric or neonatal sensor may have a particular surface area that is smaller than that of an adult sensor. Accordingly, the shielding element/s 80 are sized and shaped to be compatible with a particular size sensor 12. In another embodiment, the shielding elements 80 may be provided as an accessory, e.g., as a package including a plurality of shielding elements 80 of various sizes that may be used in conjunction with a range of sensor sizes. In such an embodiment, the shielding elements and/or the sensors 12 may include markings or sizing indicators to help caregivers use the correct shielding element 80 with the correct sensor 12.

In the depicted embodiment, a back side or non-tissue contacting surface 82 of the sensor 12 is shown. The shielding elements are configured to be applied to the non-tissue-contacting surface 82, which is exposed when the sensor 12 is applied to the tissue. The shielding element 80 may be applied concurrently with the sensor 12 or may be applied after the sensor 12 has already been in use. In certain embodiments, the sensor 12 includes a sensor body 44 portion and an adhesive layer 86 disposed about at least a portion of the perimeter 88 of the sensor body 44. For example, the adhesive layer 86 may be disposed about most of the perimeter 88 (e.g., about more than 50% of the total length of the perimeter). The adhesive layer 86 may provide all or a part of the adhesion of the sensor 12 to the tissue. That is, the contacting surface of the sensor body 44 may also have some adhesive properties. In other embodiments, the sensor 12 may also include no adhesive layer about the perimeter 88. When present, the adhesive layer 86 may include a dark or light-absorbing material to provide a first layer of defense against ambient light infiltration. However, in embodiments in which no adhesive layer 86 is present or in embodiments in which the adhesive layer 86 provides insufficient ambient light shielding, the shielding element 80 may be applied to the non-tissue contacting surface 82 and to the surrounding tissue about the sensor 12 to achieve more light shielding.

For example, a disposable medical sensor may be repositioned a number of times on the patient before being ultimately discarded. With each repositioning, the adhesive on the sensor may weaken such that the sensor body no longer closely conforms to the underlying tissue. In such instances, ambient light infiltration may interfere with accurate sensor measurements. In the present embodiments, the sensor 12 may be used in conjunction with the shielding element 80 to help the sensor body 44 conform to the tissue. Further, a new shielding element 80 may be used with each successive repositioning of the sensor 12.

The shape of a perimeter 88 of the sensor body 44 may be selected such that a perimeter 96 of the shielding element 80, when applied, extends beyond the perimeter 88 of the sensor body as well as a perimeter 98 of the adhesive layer 86 (if present) such that the shielding element adheres to both the sensor 12 and the tissue. That is, the shielding element 80 includes portions that are wider than the sensor 12, including any adhesive layers 86. In the depicted embodiment, the perimeter 96 of the shielding element 80 mimics the contours of the perimeter 98 of the adhesive layer, but is wider. In one embodiment, the perimeter 96 of the shielding element extends beyond at outermost perimeter of the sensor 12 (e.g., perimeter 98 if present or perimeter 88) at least 1 mm.

In certain embodiments, the shielding element 80 includes cutouts 100 and 102 separated by a bridge and that are sized and shaped to be close in size to lobes 110 and 112 of the sensor body 44. The bridge 104 is positioned to align with a neck 114 that provides a generally hourglass shape to the sensor body 44. Further, an operator may use the shape of the sensor body, including the contours of the first lobe 110, the second lobe 112, and the neck 114 as a guide for placement of the cutouts 100 and 102 and the bridge 104.

FIG. 4 is a side exploded view of the shielding element 80 and the sensor 12. As shown, the shielding element 80 is aligned with the sensor body 44 via the positions of the cutouts 100 and 102 and the bridge 104. In one embodiment, positioning the bridge 104 on the neck 114 of the sensor body 44 generally aligns the cutout 100 with the first lobe 110 and the cutout 102 with the second lobe 112. Once aligned, the shielding element can be adhered to the sensor 12, via at least a top surface 120 of the adhesive layer and the sensor body 44 with the adhesive surface 122 of the shielding element.

In certain embodiments, the shielding element 80 may be formed from a material that is relatively thin compared to a thickness d₁ of the sensor body 44. In particular, the sensor body 44 may be formed of multiple layers, including one or more foam layers. In contrast, the shielding element may be formed of a relatively thin paper or polymer material that is less than 50% of the thickness d₁ of the sensor body 44. A relatively thin shielding element 80 may be advantageous in embodiments in which multiple shielding elements 80 are positioned on top of one another. For example as the sensor is repositioned a new shielding element 80 may be applied over a shielding element 80 that is already in place. Removal of an adhered shielding element 80 may result in damage to the sensor body or wrinkling of the adhesive layer 86. Accordingly, an operator may reposition the sensor and place a new shielding element on top of an old shielding element 80. The addition of one or more relatively thin shielding elements 80 may have limited effect on the overall profile the sensor 12 on the tissue. In such an embodiment, the subsequently applied shielding elements 80 may be wider and/or larger such that their outer perimeters 96 extend beyond the perimeters 96 of any shielding elements 80 that are in place. Further, the size of the shielding element 80 and its outer perimeter 96 may be selected so there is enough surface area to conform to and wrap around the thickness d₁ of the sensor body 44 and extend beyond the outermost perimeter (e.g., perimeter 88 or perimeter 98) of the sensor 12 without forming a gap. In one embodiment, a sensor kit may include shielding elements with cutouts 100 and 102 that are approximately the same size but with differently-sized outer perimeters 96. The kit may include instructions to operators to apply smaller shielding elements 80 first and larger shielding elements 80 over smaller shielding elements already in place.

The adhesive layer 86 and the adhesive surface 122 of the shielding element 80 may include any adhesive material suitable for integration into medical devices (e.g., a hypoallergenic adhesive material). By way of example, suitable adhesives may include an acrylic adhesive or a hydrocolloid adhesive. Generally, hydrocolloid adhesives may provide enhanced comfort for the patient and avoid damage to the patient's skin when the sensor 12 is removed or repositioned. Further, the adhesive may be a transfer adhesive or may be a single-sided adhesive. Thus, the adhesive surfaces of the adhesive layer 86 and the adhesive surface 122 of shielding element 80 will include the adhesive material, but a top surface of the adhesive layer 86 and the shielding element 80 may not be adhesive. A release liner may also be provided to prevent the inadvertent attachment of the adhesive surface 122 or the adhesive layer 86 to a surface before the intended use of the sensor 12. The release liner may include any liner having a release material, such as a coated release paper or a release plastic film. Example release materials include polyolefins (e.g., polypropylene, high- and low-density polyethylene), polyesters (e.g., biaxially-oriented polyethylene terephthalate), polyvinyl alcohol, Kraft paper, polystyrene, or the like.

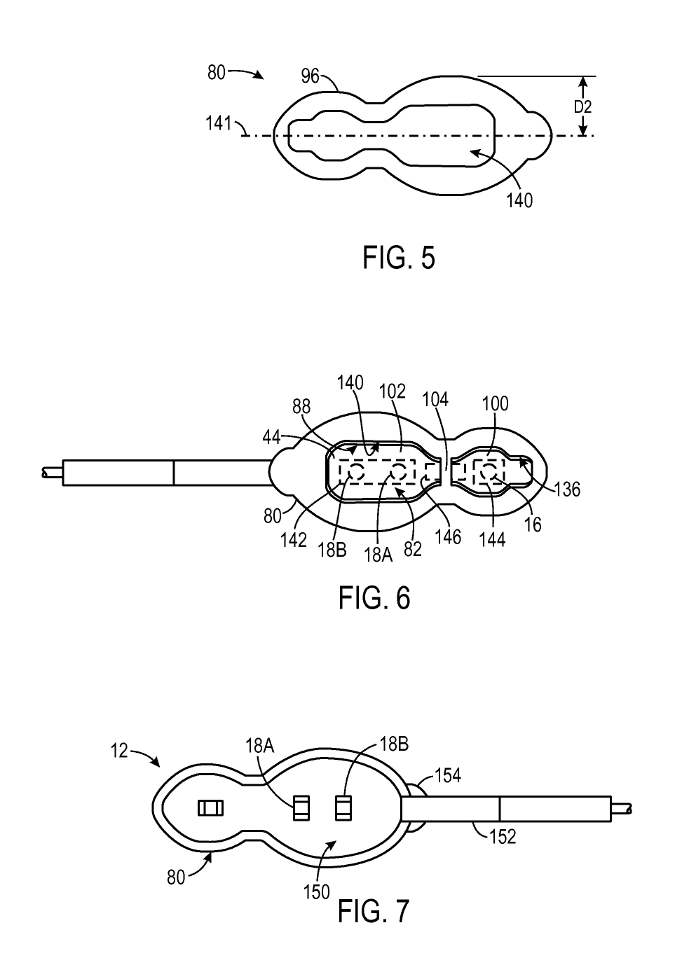

The shielding element 80 may be characterized by its surface area, which is a function of its size and the size of any cutouts. The overall size of the outer perimeter may be selected based on the tissue site of interest, the sensor size, and the patient size. FIG. 5 is an alternative embodiment of a shielding element 80 that only includes a single cutout 140. It should be understood that the shielding element 80 may include one or more cutouts, as desired. For example, the shielding element 80 may include cutouts specific to portions of a sensor that include emitters and/or detectors. In the depicted embodiment, the effect of the light infiltration may be most pronounced in and around the detector. In such an embodiment, the cutout 140, positioned near the detecting components, may provide sufficient shielding. In another embodiment, the overall size of the shielding element 80 is selected to fit within a patient's forehead. Accordingly, the shielding element 80 has an elongated shape, such as a generally rectangular, hourglass, or elliptical shape including a major axis along a longest dimension and a minor axis along a shortest dimension. In one embodiment, a largest distance d₂ from a point along a major axis 141 of the shielding element is 45 mm or less.

In one embodiment, the optical components of the sensor 12 are positioned in one or both of the first lobe or the second lobe, while the neck does not include any optical components. Accordingly, the shielding element 80, when in place, does not cover the portions of the sensor body 44 with the optical components. By providing cutouts that correspond to locations on the sensor body associated with the emitter 16 and the detectors 18A and 18B, a shielding element 80 may be configured to apply pressure unevenly across the sensor body 44. For example, as shown in FIG. 6, the interior border 136 of the cutout 100 is sized such that an area 144 around the emitter 16 is uncovered when the shielding element 80 is in place. Similarly, the interior border 140 of the cutout 102 is sized and shaped so that an area 142 around the detectors 18 is uncovered. In contrast, where the bridge 104 covers the sensor body 44, there is an area 146 that is at least in part covered by the shielding element 80. The shielding element 80, when adhered to the sensor body 44 and the tissue, applies greater pressure to the area 146 while the areas 144 and 142 are relatively unaffected by the application of the shielding element 80. In this manner, the shielding element 80 may prevent light infiltration without significantly changing the overall pressure of the sensor 12 on the tissue. Further, because the area 146 generally corresponds to the neck, the local increased pressure is provided in a more limited, i.e., thinner or smaller surface area portion.

While bandage-type or conformable sensors include flexible sensor bodies 44, the optical components, including any housing or encapsulating structures for the emitter 16 and/or detector 18, may be formed from rigid plastic materials. By limiting the pressure applied to areas directly surrounding the optical components of the sensor 12, the pressure applied by the rigid components of the sensor 12 may also be limited, thus decreasing the incidence of pressure marks on the skin as well as increasing patient comfort. Accordingly, the shielding element 80 may be configured to cover less than 50%, 40%, 30%, 20%, 10%, or 5% of the surface area of the sensor body 44 when applied to the sensor body 44 in operation. Covering less surface area of the sensor body 44 may result in less overall pressure applied to the tissue and the optical elements via the sensor body 44. In another embodiment, when the sensor 12 includes an adhesive layer 86 (see FIG. 3), the shielding element 80 may be configured to cover more than 50%, 60%, 70%, 80%, or 90% of the adhesive layer 86. For example, the adhesive layer 86 may be configured as a separate element or layer extending from the perimeter 88 of the sensor body 44. Accordingly, pressure applied to the adhesive layer 86 by the shielding element 80 may have less effect on the optical components, which are disposed on the sensor body 44 and not the adhesive layer 86.

In embodiments in which the sensor 12 is configured for pulse oximetry, the sensor 12 including the shielding element 80 may provide sufficient pressure to the tissue so that the applied pressure exceeds the typical venous pressure of a patient, but does not exceed the diastolic arterial pressure. A sensor that applies a pressure greater than the venous pressure may squeeze excess venous blood from the optically probed tissue, thus enhancing the sensitivity of the sensor to variations in the arterial blood signal. Since the pressure applied by the sensor is designed to be less than the arterial pressure, the application of pressure to the tissue does not interfere with the arterial pulse signal. Typical venous pressure, diastolic arterial pressure and systolic arterial pressure are typically less than 10-35 mmHg, 80 mmHg, and 120 mmHg, respectively, although these pressures may vary because of the location of the vascular bed and the patient's condition. In certain embodiments, the sensor may be adjusted to overcome an average pressure of 15-30 mmHg. In other embodiments, low arterial diastolic blood pressure (about 30 mmHg) may occur in sick patients. In such embodiments, the sensor may remove most of the venous pooling with light to moderate pressure (to overcome about 15 mmHg). In embodiment in which the sensor 12 is configured to detect regional saturation, the pressure of the sensor 12 on the tissue, even with a shielding element 80 in place, may be low enough so that venous blood is not squeezed from the tissue (e.g., less than 5-10 mm Hg or less than 3-5 mm Hg.

The amount of additive pressure applied by the shielding element 80 to the sensor body 44 may also be affected by the size of the cutouts. In one embodiment, the cutout 102 is slightly smaller than, the same size as, or slightly larger than the first lobe 110. When the cutout 100 is positioned entirely on the sensor body 44 such that an interior border 136 of the cutout 100 is within the perimeter 88, the pressure applied overall to the sensor body 44 may be greater. Alternatively, as shown in FIG. 6, the cutout 100 may be slightly larger than the first lobe 110 in at least one dimension such that a portion of the interior border 136 rests on a top surface 120 of the adhesive layer 86 and less of the shielding element 80 rests on the sensor body 44. Similarly, the cutout 102 may be slightly smaller than, the same size as, or slightly larger than the second lobe 112, and an interior border 140 may rest on the sensor body 44 or in part on the adhesive layer 86. In certain embodiments, to decrease the overall pressure on the sensor body 44, and, correspondingly, the areas 144 and 142, one or both cutouts 100 and 102 may be configured such that less than 50% of their interior borders 136 and 140 are adhered to the non-tissue contacting surface 82 the sensor body 44. Correspondingly, the portion of the interior borders 136 and 140 not adhered directly to the sensor body 44 may be adhered to the adhesive layer 86, when present, or directly to the tissue.

FIG. 7 is a view of a patient-contacting surface 150 of the sensor of FIG. 6. The shielding element 80 does not directly contact the surface 152 when applied, but instead wraps around a non-tissue contacting surface 82 (see FIG. 3) to form an edge of the sensor assembly. The sensor 12 is applied by placing the surface 152, on which the emitter 16 and detectors 18A and 18B are disposed, in direct contact with a patient's tissue. Accordingly, the shielding element 80 does not interfere with light emission and light detection when the sensor 12 is in operation. In one embodiment, the sensor 12 also includes a cable 152 that carries electrical connections to and from the emitter 16 and detectors 18. As shown, the shielding element may include a tab 154 that is aligned along an axis formed by the cable 152. The tab 154 may be configured to allow an operator to align the shielding element 80 correctly with the sensor 12 by aligning the tab 154 with the cable 152.

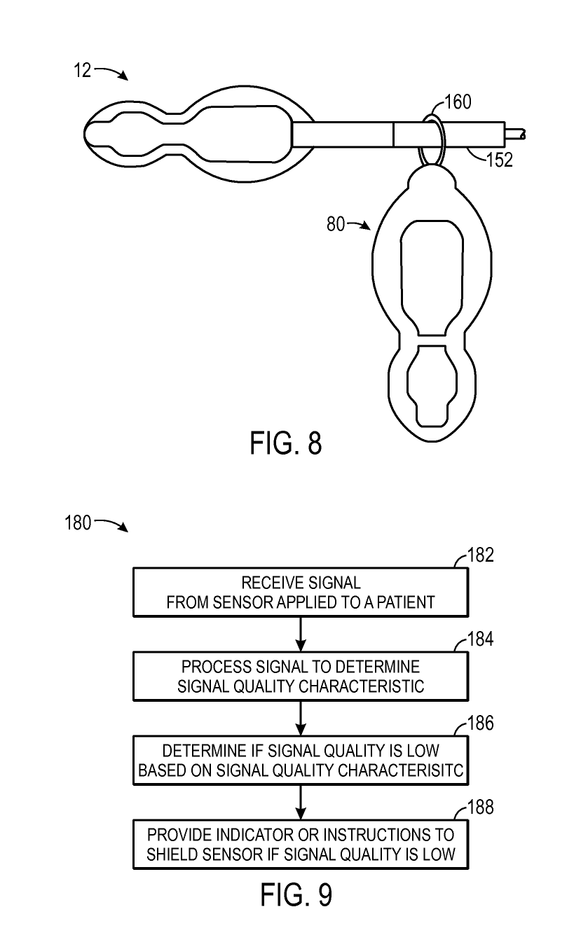

The shielding elements 80 may be packaged with the sensor 12 as a unitary assembly for the convenience of the end user. In one embodiment, the sensor 12 may include shielding elements that are coupled to or otherwise retained with the sensor assembly even when not in use. As shown in FIG. 8, the shielding element 80 may be retained on the cable 152 by a loop 160. If the loop 160 is elastic or the retaining feature is long enough, the shielding element may be placed on the sensor 12 while still in association with the cable 152. In other embodiments, the shielding element may be removed from the cable 152, for example by cutting the loop 160, before being used.

As disclosed herein, the sensor 12 and shielding element 80 may be used in conjunction with the system 10 (FIG. 1) for monitoring a physiological parameter. The shielding element 80, when in place, reduces ambient light n from impinging the detector/s 18 and interfering with measurement accuracy. In one embodiment, the shielding element may be applied as desired by a caregiver, for example, concurrently with sensor application, or upon sensor repositioning. In other embodiments, the shielding element 80 may be applied when the system 10 detects ambient light infiltration. FIG. 9 is a flow diagram 180 of a method of determining if a shielding element 80 should be applied to the sensor 12. At step 182, the system 10 or the monitor 14 receives a signal from the sensor 12. For example, for plethysmography sensors, the system 10 receives a signal representative of light detected by one or more detectors 18. Processing circuitry (e.g., processor 48) associated with the monitor 14 processes the signal and assesses one or more signal quality characteristics of the signal at step 184 and determines if the signal quality is low at step 186. If the signal quality is low, an alarm, indication, or instructions, or any combination thereof, is provided at step 188 to prompt the caregiver to shield the sensor 12. Signal quality may be assessed by assessing quality metrics such as pulse shape; pulse amplitude; modulation ratios of the red to infrared signals, known as ratio of ratios and pulse period. Moreover, other indicators that trend the consistency of pulse shapes relative to each of these additional indicators are also viable as measures of signal quality.

In another embodiment, the amount of ambient light infiltration may be assessed by comparing a detected signal from a dark period of the sensor 12, i.e., a period when the emitter is not emitting light, to a threshold. In a specific embodiment, the monitor 14 is configured to receive a dark period signal and invert the signal and combine the inverted signal with the received signals during times that the emitter is active to subtract or account for the effects of ambient light. However, such subtraction results in an overall decrease in signal amplitude, particularly when there is a significant amount of ambient light. In one in embodiment, when the detected signal during the dark period exceeds the threshold or when the combined signal falls below a desired amplitude, an indication or instructions may be provided to apply a shielding element 80. Further, the monitor 14 may also be configured to receive a user input that the shielding element 80 is in place and to perform an additional check for ambient light infiltration and/or signal quality.

Although the disclosed embodiments have been depicted using a reflectance sensor including an emitter and two detectors, it should be understood that the shielding elements 80 disclosed herein may be configured for use with other sensor configurations. For example, the shielding elements 80 may be used with transmission type sensors. In addition, the shielding elements may be used in conjunction with sensors that have a single emitter-detector pair, such as pulse oximetry sensors, as well as other emitter/detector arrangements.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A sensor assembly, comprising:
a sensor body comprising a first lobe and a second lobe coupled by a neck;
an emitter disposed on a patient-contacting side of the sensor body on the first lobe and configured to transmit light into a patient tissue;
a first detector disposed on the patient-contacting side of the sensor body on the second lobe and configured to detect a first portion of light passing through the patient tissue;
a second detector disposed on the patient-contacting side of the sensor body on the second lobe and configured to detect a second portion of light passing through the patient tissue, wherein the first detector is closer to the emitter than the second detector;
an adhesive layer disposed about at least a portion of a perimeter of the sensor body; and
a shielding element configured to be adhered to the sensor body on a surface opposing the patient-contacting surface and to the adhesive layer, wherein the shielding element, when adhered to the sensor body, extends beyond the perimeter of the sensor body and does not cover the sensor body in an area corresponding to the emitter, the first detector, and the second detector.

2. The sensor assembly of claim 1, wherein the sensor is a regional oxygen saturation sensor.

3. The sensor assembly of claim 1, wherein the emitter comprises at least one light emitting diode.

4. The sensor assembly of claim 1, wherein the shielding element extends beyond a perimeter of the adhesive layer.

5. The sensor assembly of claim 1, wherein the shielding element covers less than 20% of a surface area of the surface opposing the patient-contacting surface.

6. The sensor assembly of claim 1, wherein the shielding element comprises a first opening configured to align with the first lobe, wherein the first opening is smaller than the first lobe.

7. The sensor assembly of claim 6, wherein the shielding element comprises a second opening configured to align with the second lobe, wherein the second opening is smaller than the second lobe.

8. The sensor assembly of claim 1, wherein the shielding element comprises an opening divided into a plurality of portions by at least one bridge.

9. The sensor assembly of claim 1, wherein the at least one bridge covers at least a portion of the neck.

10. The sensor assembly of claim 1, wherein the shielding element is formed from a light-absorbing material.

11. The sensor assembly of claim 1, wherein the shielding element is thinner than the sensor body.

12. The sensor assembly of claim 1, wherein the emitter and the detector are coupled to a cable extending from the sensor body, and wherein the shielding element comprises a tab aligned with the cable.

13. The sensor assembly of claim 1, wherein the shielding element is configured to apply pressure unevenly across the sensor body such that a pressure of the sensor body against a patient's tissue is uneven.

14. The sensor assembly of claim 13, wherein a pressure applied by the perimeter of the sensor body against the tissue is greater than a pressure applied by an area corresponding to the emitter, the first detector, and the second detector when the shielding element is applied to the sensor body.

15. The sensor assembly of claim 14, wherein the pressure applied by the perimeter of the sensor body against the tissue and the pressure applied by an area corresponding to the emitter, the first detector, and the second detector when the shielding element is applied to the sensor body are less than 5 mm Hg.

16. A sensor assembly, comprising:
a sensor body;
an emitter disposed on a patient-contacting side of the sensor body;
at least one detector disposed on the patient-contacting side of the sensor body and configured to detect a first portion of light passing through the patient tissue; and
a shielding element comprising an adhesive surface configured to be adhered in part to a patient's tissue and in part to the sensor body on a surface opposing the patient-contacting surface layer, wherein the shielding element, when adhered to the sensor body, applies pressure unevenly across the sensor body such that a pressure applied to the patient's tissue by the emitter and the at least one detector is less than a pressure applied to the patient's tissue by a perimeter of the sensor body.

17. The sensor assembly of claim 16, wherein the sensor is a regional oxygen saturation sensor.

18. The sensor assembly of claim 16, wherein the shielding element comprises an opening that aligns with a location of the emitter such that the shielding element does not cover the emitter when applied to the sensor body.

19. The sensor assembly of claim 16, wherein the shielding element comprises a release liner that covers the adhesive surface and that is configured to be removed before adhering the shielding element to the sensor body.

20. The sensor assembly of claim 16, comprising a second shielding element, wherein the second shielding element has a larger adhesive surface area that the first shielding element.

21. The sensor assembly of claim 16, comprising a retention element configured to hold the shielding element on a cable coupled to the emitter and the at least one detector before the shielding element is applied to the sensor body.

22. A method comprising:
applying a sensor to a patient's tissue wherein the sensor comprises a sensor body having a first lobe and a second lobe coupled by a neck such that a first optical element disposed on the first lobe and a second optical element disposed on the second lobe are in direct contact with the patient's tissue;
receiving an indication that measurements acquired by the sensor are associated with ambient light infiltration; and
applying a shielding element in part to the sensor body on a surface opposing a patient-contacting surface and in part to the patient's tissue in an area about a perimeter of the sensor body such that the shielding element, when adhered to the sensor body, extends beyond the perimeter of the sensor body and does not cover the sensor body in an area corresponding to the first optical element and the second optical element, wherein the applying of the shielding element is only when the measurements acquired by the sensor are associated with ambient light infiltration.
